(19) United States
(12) Patent Application Publication
(10) Pub. No.: US 2003/0228311 A1
(43) Pub. Date: Dec. 11, 2003

(54) Methods for enhancing oral tolerance and treating autoimmune disease using inhibitors of interleukin-12

(76) Inventors: Warren Strober, Bethesda, MD (US); Brian Kelsall, Washington, DC (US); Thomas Marth, Kensington, MD (US)

Correspondence Address: NATIONAL INSTITUTE OF HEALTH, C/O NEEDLE & ROSENBERG, P.C., SUITE 1000, 999 PEACHTREE STREET, ATLANTA, GA, 30303, US

(21) Appl. No.: 10/421,650

(22) Filed: Apr. 21, 2003

Related U.S. Application Data
(63) Continuation of application No. 09/732,502, filed on Dec. 7, 2000, now abandoned.
Application No. 09/732,502 is a continuation of application No. 09/284,169, filed on Apr. 9, 1999, now abandoned.
Application No. 09/284,169 is a 371 of international application No. PCT/US96/16007, filed on Oct. 11, 1996.

(51) Int. Cl. A61K 39/395

(52) U.S. Cl. 424/145.1

(57) ABSTRACT

The present invention provides a method for enhancing oral tolerance to an antigen associated with an autoimmune disease in a subject having the autoimmune disease comprising orally administering to the subject an antigen associated with the autoimmune disease and administering an inhibitor of interleukin-12 in amounts sufficient to enhance oral tolerance. Also provided in the present invention is a method for treating or preventing an autoimmune disease in a subject comprising orally administering to the subject an antigen associated with the autoimmune disease and administering an inhibitor of interleukin-12 in amounts sufficient to treat or prevent the auto immune disease, thereby treating or preventing the autoimmune disease.

### BACKGROUND OF THE INVENTION

1 Field of the Invention

The present invention relates to a method for enhancing oral tolerance in a subject with an autoimmune disease, allergic disease, graft-versus-host (gvH) disease or transplantation rejection. In particular, the present invention provides a method for enhancing oral tolerance in a subject to treat an autoimmune disease, allergic disease, GvH disease or transplantation rejection comprising orally administering an antigen associated with the autoimmune disease, allergic disease, GvH disease or transplantation rejection to be treated and administering an inhibitor of interleukin-12 (IL-12) in amounts sufficient to enhance oral tolerance in the subject and treat or prevent autoimmune disease, allergic disease, GvH disease or transplantation rejection.

2. Background Art

The administration of soluble protein antigen via the oral route has been described as a means of inducing antigen-specific systemic immune tolerance (oral tolerance) [reviewed in (1) and (2)]. Recently, this method of tolerance induction has been used to suppress autoimmune diseases in animals (3-6), and is currently being tested for the treatment of autoimmune disorders such as multiple sclerosis, rheumatoid arthritis, and autoimmune uveoretinitis in humans (2, 7, 8).

Two possibly related immunological mechanisms believed to be involved in oral tolerance are clonal anergy and clonal deletion. This was shown by Whitacre et al., as well as others, who demonstrated that the administration of single high doses of oral antigen is associated with the loss of specific immune responsiveness in the absence of active T cell suppression (4, 9). In addition, Chen et al. demonstrated that the feeding of high doses of antigen multiple times results in deletion of antigen-specific T cells which is mediated by apoptotic cell death (10).

Another immunologic mechanism thought to account for tolerance following antigen feeding is active suppression. It has been found that feeding of low dose antigen to normal animals results in the activation of CD4⁺ and CD8⁺ T cells producing cytokines capable of suppressing immune responses (9-12). Weiner and colleagues have demonstrated that transforming growth factor beta (TGFβ) plays a critical role in the suppression of experimental allergic encephalomyelitis (EAE) after the feeding of myelin basic protein (11, 13, 14). These studies suggest that suppressor T cells arise in the mucosal immune system and migrate to systemic sites where upon antigen-specific reactivation they release TGFβ and perhaps other suppressive cytokines. These cytolines, in turn, down regulate T cell responses not only to specific (fed) antigen, but also to other antigens (15, 16). Such “bystander” suppression is the basis for possible treatment of autoimmune diseases with the oral administration of autoantigens that may have a minor role in disease induction (e.g. oral insulin for diabetes mellitus) as well as for the possible treatment of allergic disease, graft-versus-host (GvH) disease or transplant rejection.

Interleukin-12 is a recently characterized cytokine with unique structure and pleiotropic effects (40-43). It consists of two disulfide-linked subunits, p40 and p35, that form functionally active p40/p35 heterodimers or inhibitory p40 homodimers. IL-12 is produced mainly by macrophages/monocytes and can be efficiently induced by intracellular parasites, bacteria and bacterial products. Functional studies have shown that IL-12 enhances cytolytic activity of natural killer (NK) cells and macrophages and induces, in synergism with the B7/CD28 interaction, cytokine production and proliferation of activated NK cells and T cells (44). Furthermore, IL-12 plays a pivotal role in Th1 T cell differentiation and induces naive T cells to produce IFN-γ. As a result of this ability to drive T cell responses to the Th1 phenotype, administration of IL-12 has been shown to be an effective treatment of mice with established parasitic infections, which elicit a Th2 T cell response (45,46).

Despite recent advances in the understanding and clinical use of oral tolerance, the immune mechanisms responsible for this phenomenon have not been clearly established. Furthermore, enhancement of the therapeutic effect could be critical to the ultimate clinical usefulness of oral tolerance to treat autoimmune disease, allergic disease, GvH disease or transplantation rejection. The present invention overcomes previous shortcomings in the clinical use of oral tolerance for treating or preventing autoimmune disease, allergic disease, GvH disease or transplantation rejection by providing a method for enhancing oral tolerance in a subject comprising orally administering an oral tolerance-inducing antigen and administering an inhibitor of IL-12.

### SUMMARY OF THE INVENTION

The present invention provides a method for enhancing oral tolerance to an antigen associated with an autoimmune disease in a subject having the autoimmune disease comprising orally administering to the subject an antigen associated with the autoimmune disease and administering an inhibitor of IL-12 in amounts sufficient to enhance oral tolerance.

Also provided in the present invention is a method for treating or preventing an autoimmune disease in a subject comprising orally administering to the subject an antigen associated with the autoimmune disease and administering an inhibitor of IL-12 in amounts sufficient to treat or prevent the autoimmune disease, thereby treating or preventing the autoimmune disease.

Various other objectives and advantages of the present invention will become apparent from the following description:

### DETAILED DESCRIPTION OF THE PREFERRED EMBODIMENTS

The present invention may be understood more readily by reference to the following detailed description of specific embodiments and the Examples included herein. As used in the claims, “aa” can include multiples.

The present invention provides a method for enhancing oral tolerance to an antigen associated with an autoimmune disease, an allergic disease, GvH disease or transplantation rejection in a subject having any of these diseases comprising orally administering to the subject an antigen associated with the autoimmune disease, allergic disease, GvH disease or transplantation rejection and administering an inhibitor of IL-12 in amounts sufficient to enhance oral tolerance. Any animal which is subject to autoimmune disease, allergic disease, GvH disease or transplantation rejection can be treated by this method although humans are the primary therapeutic target. Examples of nonhuman subjects that can be treated by the methods taught herein can include, but are not limited to, mice, rats, rabbits, dogs, cats, non-human primates, as well as any other species now known or later discovered to manifest an autoimmune disease, allergic is disease, GvH disease or transplantation rejection.

As used herein, “oral tolerance” refers to an antigen-induced immune response initiated by the oral administration of a soluble protein antigen and resulting in systemic unresponsiveness to the administered antigen. Such unresponsiveness may be specific for the administered antigen or may be antigen-non-specific as a result of production of an antigen-non-specific suppressor substance such as TGFβ. Thus, enhancement of oral tolerance as used herein means increasing systemic unresponsiveness induced by oral antigen. The enhancement of oral tolerance induced by an oral antigen can be by any means by which clonal deletion or the production of TGFβ is augmented (1,2,65). Such means can also include, but are not limited to, administering TGFβ directly to the subject; administering an inhibitor of interferon-gamma to the subject; administering an-antibody to the interferon-gamma receptor, which prevents binding of interferon-gamma, to the subject; administering an inhibitor of tumor necrosis factor-alpha to the subject; administering an antibody to the tumor necrosis factor-alpha receptor, which prevents binding of tumor necrosis factor-alpha, to the subject; administering an inhibitor of interferon-alpha to the subject; administering an antibody to the interferon-alpha receptor, which prevents binding of interferon-alpha, to the subject; administering interleukin-10 (IL-10) to the subject; or any combination of these means, as well as other means or combinations of means now known or identified in the future to augment clonal deletion or the production of TGFβ in a subject to enhance oral tolerance induced by an oral antigen.

Also as used herein, autoimmune disease describes a disease state or syndrome whereby a subject's body produces a dysfunctional immune response against the subject's own body components, with adverse effects. This may include production of B cells which produce antibodies with specificity for all antigens, allergens or major histocompatibility (MHC) antigens or production of T cells bearing receptors recognizing self components and producing cytokines that cause inflammation. Examples of autoimmune diseases include, but are not limited to, ulcerative colitis, Crohn's disease, multiple sclerosis, rheumatoid arthritis, diabetes mellitus, pernicious anemia, autoimmune gastritis, psoriasis, Bechet's disease, idiopathic thrombocytopenic purpura, Wegener's granulomatosis, autoimmune thyroiditis, autoimmune oophoritis, bullous pemphigoid, pemphigus, polyendocrinopathies, Still's disease, Lambert-Eaton myasthenia syndrome, myasthenia gravis, Goodposture's syndrome, autoimmune orchitis, autoimmune uveitis, systemic lupus erythematosus, Sjogren's syndrome and ankylosing spondylitis (1,2,69,70,73-77,85-89), as well as any other autoimmune disease now known or discovered in the future.

Also as used herein, allergic disease describes a disease state or syndrome whereby the body produces a dysfunctional immune response composed of immunoglobulin E (IgE) antibodies to environmental antigens and which evoke allergic symptoms. Examples of allergic diseases include, but are not limited to, asthma, ragweed pollen hayfever, allergy to food substances and allergic reactions (54-60,78-80). Also as used herein, GvH disease describes a disease state or syndrome whereby an immune response is initiated by grafted cells and is directed against the subject's body with adverse effects. Examples of GvH disease include, but are not limited to, acute and chronic GvH disease following bone marrow transplant (64). Transplantation rejection describes a disease state or syndrome whereby the transplant recipient's body produces an immune response against the engrafted tissue, resulting in rejection. Transplantation rejection can occur, for example, with kidney, heart, lung or liver transplants as well as with any other transplanted tissue (61-63, 81-84).

An antigen associated with an autoimmune disease is a self antigen or an antigen that cross reacts with a self antigen to which the body produces a dysfunctional immune response that causes disease. An antigen associated with an allergic disease is an environmental antigen (allergen) which induces an individual with an appropriate genetic/environmental background an IgE response which mediates mast cell/basophil activation, causing allergic symptoms. An antigen associated with GvH disease or transplantation rejection comprises an MHC to which grafted cells or host-cells react and cause adverse effects. These antigens or portions of antigens can be administered to the subject by oral or nasal route to induce oral tolerance. For example, such antigens can be, but are not limited to, myelin basic protein, collagens, allergens, insulin, Fel-d-1, cells expressing MHC antigens (54-64, 78-89), as well as any other antigen now known or identified in the future to be functional in inducing or enhancing oral tolerance in a subject. The antigen of this invention can be a single type of antigen or a combination of types of antigens. The antigen of this invention can either be from the species to which it is administered or it can be a homologous antigen from a different species.

The inhibitor of IL-12 can be an antibody, either polyclonal or monoclonal, that is specifically reactive with interleukin-12 or a receptor which binds IL-12. Other inhibitors of IL-12 can include, but are not limited to, p40 IL-12 homodimers, inhibitors of IL-12 production, such as IL-10, TGFβ, dimerized complement components, C₃B, C₃Bi, antibodies to CD46 or complement receptor (CR3), as well as any other compound or reagent now known or later discovered which acts to inhibit the activity or production of IL-12.

The antibodies of this invention can be from any source. However, to reduce the immunogenicity of the immunoglobulins themselves, antibodies are preferably of human origin or are antibodies generated in other species and “humanized” for administration in humans as described in the Examples provided herein. The antibodies of this invention can be fragments which retain the ability to bind their specific antigens are also contemplated for this invention. For example, fragments of antibodies which maintain IL-12 binding activity, as well as fragments of IL-12 which maintain IL-12 binding activity (e.g., homodimer formation) but which function to inhibit IL-12 activity and thus reduce the TGFβ-inhibiting effects of IL-12, are included within the meaning of the term “antibody.” Such antibodies and fragments can be made by techniques known in the art and screened for specificity and activity according to the methods set forth in the Examples herein. For example, general methods for producing antibodies can be found in Harlow and Lane (48).

The present invention also provides a method for treating or preventing an autoimmune disease, allergic disease, GvH disease or transplantation rejection in a subject comprising orally administering to the subject an antigen associated with the autoimmune disease, allergic disease, GvH disease or transplantation rejection and administering an inhibitor of interleukin-12 in amounts sufficient to treat or prevent the autoimmune disease, allergic disease, GvH disease or transplantation rejection, thereby treating or preventing the autoimmune disease, allergic disease, GvH disease or transplantation rejection. The autoimmune disease, allergic disease, GvH disease or transplantation rejection can be treated or prevented by inducing oral tolerance in a subject in which the administration of an oral antigen alone may not be sufficient to induce oral tolerance or by enhancing an oral tolerance already induced by the administration of an oral antigen. Administration of the oral antigen includes the subject simply ingesting (eating) the antigen or the antigen can be provided.

The antigen associated with an autoimmune disease is orally administered to the subject in a pharmaceutically acceptable carrier. Suitable carriers for oral administration of the antigen include one or more substances which may also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pyrogen free saline, pharmaceutically accepted oils, or a mixture of any s of these. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel. The antigen may be contained in enteric coated capsules that release the antigen into the intestine to avoid gastric breakdown.

The antigen associated with an autoimmune disease, allergic disease, GvH disease or transplantation rejection and the inhibitor of IL-12 can be administered to the subject in amounts sufficient to enhance oral tolerance and to treat or prevent autoimmune disease, allergic disease, GvH disease or transplantation rejection. Optimal dosages used will vary according to the individual being treated and antigen being used. Typically, for treatment of humans, antigen associated with an autoimmune disease, allergic disease, GvH disease or transplantation rejection would be administered orally in a dosage range between 0.0001 and 1.5 mg/kg/body weight/day with a preferred dosage range of 0.01-0.5 mg/kg/day and most preferred dosage or 0.1 mg/kg/day. Antigens can be administered every other day for from one week to several years. Administration of the antigen can be stopped completely following a prolonged remission or stabilization of disease signs and symptoms and readministered following a worsening of either the signs or symptoms of the disease, or following a significant change in immune status, as determined by routine follow-up immunological studies well known to a clinician in this field (e.g., a return to significant reactivity of immune cells to a particular suspected or known disease-causing antigen or to a particular tolerogen (71-89).

In the present invention, the inhibitor of IL-12 can be orally or parenterally administered in a carrier pharmaceutically acceptable to human subjects. Suitable carriers for oral administration of the inhibitor of IL-12 can include one or more of the substances described above for oral administration of antigen. For parenteral administration of the inhibitor, a sterile solution or suspension is prepared in saline that may contain additives, such as ethyl oleate or isopropyl myristate, and can be injected, for example, into subcutaneous or intramuscular tissues, as well as intravenously. The inhibitor of IL-12 may be contained in enteric coated capsules that release the inhibitor into the intestine to avoid gastric breakdown.

Alternatively, the inhibitor of IL-12 may be microencapsulated with either a natural or a synthetic polymer into microparticles 4-8 μm in diameter, which target intestinal lymphoid tissues and produce a sustained release of inhibitor for up to four weeks (47,53).

In addition to the oral administration of antigen to a human subject, antibodies to IL-12, in soluble form, would typically be administered parenterally in a single dosage of between 1 mg and 100 mg/kg of body weight, with a preferred dosage range of 5-50 mg/kg and most preferred dosage of between 10 and 20 mg/kg. Subjects can be given antibodies to IL-12 as a single injection each week for between one and 52 weeks. For oral administration, 500 mg to 1000 mg of antibodies to IL-12 can be given P.O. For administration of antibodies to IL-12 in particulate form, 500 mg to 1000 mg can be microencapsulated as described for slow release over a four to eight week period.

The amount of oral antigen and inhibitor of IL-12 administered will vary among individuals on the basis of age, size, weight, condition, etc. One skilled in the art will realize that dosages are best optimized by the practicing physician and methods for determining dosage are described, for example, in Remington's Pharmaceutical Sciences (49).

The efficacy of administration of a particular dose of an oral antigen and an inhibitor of IL-12 in enhancing oral tolerance in a human subject having an autoimmune disease, allergic disease, GvH disease or transplantation rejection can be determined by evaluating the particular aspects of the medical history, the signs, symptoms and objective laboratory test that have a documented utility in evaluating disease activity. These signs, symptoms and objective laboratory tests will vary S depending on the particular disease being treated or prevented as will be well known to any clinician in this field. Examples of such methods include, but are not limited to the following:

(1) Autoimmune disease (e.g., multiple sclerosis): The severity and number of attacks, or for continuously progressive disease, the worsening of symptoms and signs, the cumulative development of disability, the number or extent of brain lesions as determined by magnetic resonance imaging and the use of immunosuppressive medications (76,77).

(2) Allergic disease (e.g., asthma): The number and severity of attacks as determined by symptoms of wheezing, shortness of breath and coughing. The measurement of airway resistance by the use of respiratory spirometry, the extent of disability and the dependence on immunosuppressive medications or bronchodilators (78-80).

(3) GvH disease or transplant rejection (e.g., rejection of lung transplant): The signs of acute and chronic rejection can include symptoms such as, for example, shortness of breath or decreased exercise tolerance. Other parameters for determining efficacy can be measurement of arterial blood gases, determination of A-a gradients, evaluation of chest X-rays and dependence on immunosuppressive medications (81-84).

Once it is established that disease activity is significantly improved or stabilized by a particular treatment, specific signs, symptoms and laboratory tests will be followed along with a reduced or discontinued treatment schedule. If disease activity recurs, based on standard methods of evaluation of the particular signs, symptoms and objective laboratory test for a particular disease, treatment can be reinitiated.

The efficacy of administration of a particular dose of an oral antigen and an inhibitor of IL-12 in treating an autoimmune disease, allergic disease, GvH disease or transplantation rejection in a subject diagnosed as having an autoimmune disease, allergic disease, GvH disease or transplantation can be determined by standard methods of evaluation of the particular signs, symptoms and objective laboratory test for a particular disease, as known in the art (75-89). If 1) a subject's frequency or severity of recurrences is shown to be improved, 2) the progression of the disease is shown to be stabilized, or 3) the need for use of other immunosuppressive medications is lessened, based on a comparison with an appropriate control group and knowledge of the normal progression of disease in the general population or the particular individual, then a particular treatment will be considered efficacious. Additionally, the efficacy of administration of a particular dose of an oral antigen and an inhibitor of IL-12 in preventing an autoimmune disease, allergic disease, GvH disease or transplantation rejection in a subject not known to have an autoimmune disease, allergic disease, GvH disease or transplantation rejection but known to be at risk of developing an autoimmune disease, allergic disease, GvH disease or transplantation rejection can be determined by evaluating standard signs, symptoms and objective laboratory tests, as would be known to one of skill in the art, over time. This time interval may be large, with respect to the development of autoimmune or allergic diseases (years/decades) or short (weeks/months) with respect to the development of GvH disease or transplantation rejection. The determination of who would be at risk for the development of an autoimmune disease or allergic disease would be made based on current knowledge of the known risk factors for a particular disease familiar to a clinician in this field, such as a particularly strong family history of disease. For GvH disease and transplantation rejection, patients undergoing transplant procedures would be considered at risk for the development of these diseases.

The present invention is more particularly described in the following examples which are intended as illustrative only since numerous modifications and variations therein will be apparent to those skilled in the art.

### EXAMPLES

Induction of Tolerance in OVA-TCR Transgenic Mice

OVA-TCR transgenic mice in a BALB/c background, clone DO11.10, that recognizes the 323-339 peptide fragment of OVA in the context of IA^{d} (17), were kindly provided by Dennis Y. Loh (Howard Hughes Medical Institute, Washington University, St. Louis, Mo.). Syngeneic BALB/c mice were obtained from the National Cancer Institute (Bethesda, Md.). Transgenic and BALB/c mice were housed in the NIAID Twinbrook II animal facility (Rockville, Md.) and in the NIH Building 10A animal facility, respectively, under standard animal housing conditions.

Female transgenic mice 6 to 12 weeks of age were fed 10 mg, 100 mg, or 250 mg of OVA protein (Sigma, St. Louis, Mo.) dissolved in 0.5 ml phosphate buffered saline (PBS) or PBS alone once or every other day for three doses by intragastric intubation. Mice were sacrificed three days after the last feeding. Control mice were fed PBS since initial studies demonstrated that cells from mice fed PBS and from mice fed high doses of control protein (human serum albumin, Sigma) showed identical proliferation and cytokine responses after OVA restimulation in vitro. In other experiments, mice were given subcutaneous injections of 50 μg OVA [dissolved in 25 μl PBS and emulsified in the same volume of complete Freund's adjuvant (CFA, Sigma)] into the right hind footpad five days after the last feeding and sacrificed six days after the injection. Footpad swelling was determined by measuring the specific increment in footpad thickness with a Mitutoyo micrometer (specified minimum reading 0.001 inches and defined closing pressure at reading point; Thomas Scientific, Swedesboro, N.J.).

In Vivo Administration of Anti-IL-12

To evaluate effects of anti-IL-12, mice receiving three doses of 250 mg OVA orally, mice fed PBS three times, and unfed mice were given intravenous injection of 0.75 mg of a rat monoclonal antibody to murine IL-12 or, as a control, 0.75 mg of a rat anti-mouse IgG (Sigma). Neutralizing anti-IL-12 antibodies were purified from ascites of nude mice injected intraperitoneally with the C17.8 hybridoma cell line (kindly provided by Dr. G. Trinchieri, The Wistar Institute, Philadelphia, Pa.) using the E-Z-Sep antibody purification procedure (Pharmacia, Piscataway, N.J.). The treatment protocol was as follows: day 0 and 4: injection of anti-IL-12; day 1, 3 and 5: feeding of 250 mg of OVA; day 8: sacrifice of mice and cell isolation.

Media

Cell culture medium (cRPMI) consisted of RPMI 1640 (Whittaker, Walkersville, Md.) supplemented with 75 mM HEPES, penicillin (100 U/ml), streptomycin (100 μg/ml), 2 mM L-glutamine, 0.05 mM 2-mercaptoethanol (Sigma), 5% NCTC-109 media (Gibco, Grand Island, N.Y.), and 10% heat-inactivated fetal calf serum (Whittaker). Iscove's modified Dulbecco's medium (cIMDM) was supplemented as cRPMI, except that only 1% of NCTC-109 media was added.

Enrichment of T Cells and Dendritic Cells

Single cell suspensions were prepared aseptically from peripheral (inguinal, popliteal), and mesenteric lymph nodes (MLN) and spleens of transgenic mice by mechanical means. For preparations of cells from the Peyer's patches (PP), the latter were dissected from the small bowel wall and digested in cIMDM supplemented with collagenase (400 Units/ml, Collagenase D, Boehringer Mannheim, Indianapolis, Ind.) and DNASE (10 μl/ml, DNASE I, Boehringer Mannheim) for 30 minutes in a 37° C. water bath. Cells then were passed through a 40 μm nylon mesh (Falcon, Franklin Lakes, N.J.). The treatment of splenocytes with collagenase, in a fashion identical to that of PP, did not affect their ability to proliferate or to produce cytokines.

Spleen or PP cells enriched for T cells were obtained using a negative immunoaffinity selection technique, in which cells were passed over an anti-mouse Ig coated glass bead column (Isocell mouse T cell column, Pierce, Rockford, Ill.) according to manufacturer's instructions; the yield of CD3 positive cells when analyzed by flow cytometry was approximately 85%.

Spleen cells from BALB/c mice enriched for dendritic cells (DC) were prepared as previously described via plastic adhesion (18). In brief, splenic tissue was digested with collagenase and DNASE I in cIMDM, and plated in cRPMI on a plastic tissue culture dish (Falcon) for 60 min at 37° C. Non-adherent cells were removed by washing with warm PBS. The adherent DC-enriched population was cultured and antigen-pulsed in cRPMI supplemented with submaximal doses of antigen (1 mg OVA/ml) for approximately 20 hours. This was followed by washing off the non-adherent, DC-enriched cell population with warm PBS. The DC population thus obtained was >70 % CD11c positive as determined by flow cytometric analysis.

Flow Cytometry

Flow cytometry of either freshly isolated whole cell preparations, purified T cells, or DC was carried out using standard procedures in conjunction with one or more of the following anti-murine antibodies: CD3 (clone 2C11), CD4 (RM4-4), CD45R (B220; RA3-6B2), all purchased from Pharmigen (San Diego, Calif.); clonotypic antibody KJ1-26 (kindly provided by Drs. K. Nakayama and D. Loh, Howard Hughes Medical Institute, St. Louis, Mo.), anti-CD11c (N418) monoclonal antibody (American Type Culture Collection, ATCC, Rockville, Md.) and PE-labeled goat F(ab′)² anti-hamster IgG (Caltag, San Francisco, Calif.). The analysis was done on a Becton Dickinson (San Jose, Calif.) FACScan flow cytometer in association with Lysis II or Cellquest software.

Assessment of Proliferation and Cytokine Production

Whole cell preparations (1×10⁵/well) or purified T cells (1×10⁵/well) were cultured in triplicates in 96 well round bottom plates (Nunc, Naperville, Ill.) with submaximal doses of OVA (1 mg/ml), or with 2×10⁴ enriched, OVA-pulsed DC, respectively, in a total volume of 200 μl cRPMI per well for the indicated time points. For the last 8 hours of cell culture 1 μCi of [³H]-thymidine (Amersham, Arlington Heights, Ill.) was added, and after harvesting, the proliferation of cells was determined by measuring [³H]-thymidine incorporation in a scintillation counter. In some cultures chicken anti-TGFβ (R+D Systems, Minneapolis, Minn.; final concentration 10 μg/ml) or normal chicken immunoglobulin (R+D Systems, 10 μg/ml) were added.

Culture supernatants frozen at indicated time points after initiation of culture were tested for the presence of IL-2, IL-10 and IFN-γ using an antigen capture enzyme-linked immunosorbent assay (ELISA) employing antibodies (capture and secondary biotinylated antibodies) purchased from Pharmigen. Standards consisted of recombinant murine (rm) IL-2, rmIFN-γ (Genzyme, Cambridge, Mass.), and rmIL-10 (Pharmigen). Concentrations of IL-4 in tissue culture fluid were determined using a murine IL4 ELISA kit (Endogen, Boston, Mass.).

Total TGFβ released from cells in culture was assayed by the following method: Cells were cultured for 72 h in serum free media consisting of RPMI supplemented with 2 mM glutamine and Nutridoma SP [1% (vol/vol), Boehringer Mannheim]. Total, activated TGFβ (19) was measured by sandwich ELISA in 100 μl of culture supernatant after acidification with 10 μl 1M HCl for 60 min at 4° C. and neutralization with 10 μl of 1M NaOH. For the sandwich ELISA a monoclonal anti-TGFβ 1, 2, 3 antibody (Genzyme) and a polyclonal chicken anti-TGFβ antibody (R+D Systems) were used as capture and secondary antibody, respectively. Recombinant human TGFβ2 (Genzyme) was used as standard.

Detection and Quantitation of Apoptotic Cells

For in situ detection of apoptosis, organs were dissected, placed in freezing media (OCT, Miles, Elkhart, Ind.) and frozen in 2-Methylbutane and dry ice. 10 μm sections of the tissue were placed on silanated slides, dried, and fixed in 10% neutral formalin. Tissue sections were placed in PBS containing 0.1 % bovine serum albumin (Sigma), and endogenous peroxidase activity was quenched by 5 min incubation with 2% H₂O₂. The labeling of degraded DNA specific for apoptotic cells was performed using a modification of the terminal deoxynucleotidyl transferase-mediated dUTP nick end labeling (TUNEL) technique by applying the Oncor ApopTag™ peroxidase system (Oncor, Gaithersburg, Md.) according to the manufacturer's instructions. Briefly, residues of digoxigenin-labeled nucleotide were added catalytically to the ends of DNA fragments by terminal deoxynucleotidyl transferase thus forming novel random heteropolymers; peroxidase-labeled anti-digoxigenin antibodies followed by the chromogen 3,3′-diaminobenzidine were then added to generate visible signals from apoptotic cells.

The quantitation of apoptotic cells by the TUNEL technique (using ApopTag fluorescein system, Oncor) and simultaneous determination of surface antigens (antibodies to KJ1-26 and CD45R) on freshly isolated cells and cultured cells was carried out by dual color flow cytometry.

Statistical Analysis

Results represent mean values (±) standard deviations (S.D.) where applicable. Statistical significance of differences was determined by the Student's t-test.

Feeding of OVA-TCR Transgenic Mice With OVA Protein Results in Systemic tolerance and Primes for IFN-\Responses in Peyer's Patches

In initial studies, the effects of oral antigen administration on immune responses in various lymphoid tissues of OVA-TCR transgenic mice were established. Accordingly, OVA-TCR transgenic mice were fed OVA protein (10, 100, or 250 mg) either once or three times, and three days after the last feeding, proliferative responses of cell populations from the spleen, peripheral (inguinal) lymph nodes, mesenteric lymph nodes (MLN), or PP were compared to those of control mice. Proliferative responses of whole spleen cells from mice fed 10 or 100 mg of antigen were similar to those from control mice, but spleen cell responses from mice fed 250 mg were significantly reduced. Similar results were obtained with purified spleen T cells stimulated with OVA-pulsed dendritic cells (DC) and with whole peripheral lymph node cells. Whole MLN cells displayed a dose dependent decrease in proliferative responses and demonstrated highly decreased proliferative responses even at the lowest dose given (10 mg). In contrast, whole PP cell proliferative responses were unchanged or even increased after administration of single high oral antigen doses (10, 100, or 250 mg), and the reduced response seen after repeated feedings of high doe OVA (3×250 mg) was not significant.

The production of cytokines (IL-2, IFN-γ, and IL4) by cells from lymphoid tissues of mice fed three doses of 250 mg OVA was determined, as this regimen induced significant systemic tolerance, i.e. suppression of spleen T cell proliferative responses. Upon restimulation in vitro with either OVA or OVA-pulsed DCs, IL-2, IFN-γ, and IL-4 production by whole spleen cells and spleen T cells was shown to be suppressed when compared to unfed controls. In contrast, IL-2 and IL-4 responses in the PP were similar to controls and the IFN-γ response was significantly elevated (5-fold) above controls. Cytokine production by whole MLN cells was very low or undetectable for every feeding regimen.

The different responses seen in the PP and spleen following oral antigen feeding were not due to differences in the proportion of CD4⁺ T cells in each tissue that express the fully rearranged transgenic TCR, since equivalent proportions of CD4⁺ T cells from each tissue stained with the monoclonal clonotype-specific antibody, KJ1-26 (20). Thus, 79% and 84% of CD4 +T cells from the PP andu spleen, respectively, were KJ1-26⁺, when analyzed by flow cytometry. The total percentage of CD4⁺ cells of total cells isolated was 29% for the PP and 34% for the spleen. For the fed mice, the percentages of CD4⁺ T/KJ1-26⁺ were 71.2% and 74.9%, for the PP and spleen respectively.

Taken together, these data suggested that high dose oral OVA administration to OVA-TCR transgenic mice primes cells for enhanced IFN-γ production in the Peyer's patch (PP), but cellular unresponsiveness (tolerance) in the peripheral tissues. Systemic administration of anti-IL-12 to fed animals enhances oral tolerance Antibodies to IL-12 were administered intravenously to OVA-TCR-transgenic mice simultaneous with antigen feeding (3×250 mg OVA) and proliferative responses of cells were determined over time in culture. The proliferative responses vary depending on what time point in culture is chosen for comparison. In the first 96 hours in culture, the PP cells from the fed, fed+anti-IL-12 and the control mice all demonstrate increases in proliferation over that time period. At later time points in culture, i.e. after 96 hours, there is a decline in proliferation of cells from all s groups and from all organ systems in culture. However, a more rapid and significant decrease in proliferation in PP and MLN cells was found in mice fed OVA and given systemic anti-IL-12. In further studies, this rapid decline in thymidine incorporation was demonstrated to be inversely proportional to the number of T cell undergoing apoptosis in vitro as determined by two color flow cytometry for apoptotic, KJ1-26⁺ cells (Table II). To control for the effects of anti-IL-12 alone, anti-IL-12 was administered to mice fed only PBS, resulting in no significant differences of proliferative responses when compared to controls.

Taken together, these data demonstrate that the proliferative responses in the PP are not suppressed in the first 96 hours of culture by feeding or feeding+anti-IL-12 treatment, but that OVA feeding+anti-IL-12 treatment results in an accelerated fall in proliferation after 96 hours which is due to enhanced apoptosis. In contrast, proliferative responses of cells from more peripheral organs, i.e. the spleen and PLNs, are dramatically suppressed in the first 96 hours of culture in the fed and the fed+anti-IL-12 treated groups, but proliferation declines at similar rates in all groups after 96 hours in culture. Since feeding OVA to OVA-TCR transgenic mice induced such strong suppression of peripheral proliferative responses, any potential enhancement of this suppression by systemic anti-IL-12 treatment was difficult to determine. Thus, while lower peak proliferative responses were found in spleen, MLN, and PLN cells from the OVA-fed+anti-IL-12 versus the OVA-fed groups, this difference reached statistically significance only for cells from the PLN.

The effect of oral antigen feeding in the presence and absence of anti-IL-12 on secondary proliferative responses following OVA rechallenge in vivo was also determined. In these studies, mice were either fed PBS or OVA (3×250 mg), or fed PBS or OVA (3×250 mg) plus systemically administered anti-IL-12 as in previous protocols. Five days after the last oral feed, the mice were administered OVA (50 ug) in complete Freund's adjuvant (CFA) in the footpad and six days later the cells from the draining popliteal LNs were harvested and cultured in vitro with OVA. Due to difficulties in demonstrating the priming of T cells from PLT in TCR-transgenic mice by systemic antigen administration, since TCR-T cells already respond with such high levels of proliferation in primary responses, it is less possible to determine any enhancement due to maturation. Therefore, the effect of OVA rechallenge at a peripheral site with predictable antigen draining was examined. The popliteal LN responses following immunization of the footpad with OVA/CFA demonstrated a typical secondary proliferative response. Thus, proliferation of OVA-stimulated LN cells from PBS-fed mice without CFA/OVA immunization peaked at 96 hours of culture and reached a maximum mean of 126,338 cpm, while LN cells from PBS-fed, immunized mice had earlier (maximum at 48 hours) and higher (mean of 231,670 cpm) peak proliferative responses. In both of these cases, feeding and feeding with systemic anti-IL-12 resulted in a statistically significant suppression of proliferation. In addition, the responses of the OVA-fed+anti-IL-12-treated mice were significantly lower than the responses with OVA feeding alone. While the administration of anti-IL-12 alone to PBS-fed mice (i.e., mice not orally tolerized) led to some suppression of popliteal LN proliferative responses, by an as yet unknown mechanism, the degree of enhancement of oral tolerance could not be attributed to direct inhibitory effects of anti-IL-12 alone.

Anti-IL-12 Treatment of OVA-Fed Mice is Associated With Increased TGF≧ Production

To investigate the mechanisms of oral tolerance in the mice fed OVA and those fed OVA and administered anti-IL-12, cells from the PP and spleen were examined for the ability to produce cytokines, particularly inhibitory cytokines that might mediate oral tolerance. In these studies, OVA-TCR transgenic mice were fed PBS or OVA (3×250 mg), or fed PBS or OVA (3×250 mg) and treated systemically with anti-IL-12. Three days after the last feeding, whole spleen and PP cells were stimulated in vitro with OVA, or purified spleen and PP T cells were stimulated with OVA-pulsed DCs and cytokine secretion into the culture supernatant was measured by cytokine-specific ELISAs. IL-2 production by splenocytes and spleen T cells was suppressed with OVA feeding and more significantly suppressed with the addition of anti-IL-12. Neither regimen had effects on IL-2 production by PP cells. IFN-γ production by whole spleen cells and spleen T cells was suppressed by OVA feeding, however, as mentioned above, PP whole cells and PP T cells produced enhanced levels of IFN-65 following OVA feeding. IFN-γ secretion was highly suppressed in all cell populations in the fed plus anti-IL-12 treated mice. In contrast to the differential effects of OVA-feeding and OVA-feeding plus anti-IL-12 treatment on the cytokine responses of the spleen and PP cells discussed thus far, IL-4 secretion was similar in spleen and PP cell cultures. In fed plus anti-IL-12 treated mice, IL4 secretion was increased when compared to OVA feeding alone, but this level was not higher than in control (unfed) mice. Anti-IL-12 treatment also affected IL-10 secretion. In cultures of whole spleen cells and whole PP cells, but not in cultures of purified spleen T cells of fed plus anti-IL-12 treated mice, IL-10 secretion was considerably increased compared to fed-only mice. The fact that IL-10 was not increased in the cultures of purified spleen T cells suggests that non-T cells were the source of the IL-10.

A different picture was obtained with respect to TGFβ production. In this case, spleen and PP whole cells and purified T cells from OVA-fed plus anti-IL-12 treated mice produced increased amounts of TGFβ compared to OVA-fed-only and control mice; this effect was particularly evident in PP cells, where it was clear that cells from OVA-fed-only mice produced no more TGFβ than controls, but cells from OVA-fed plus anti-IL-12 treated mice produced very high levels of TGFβ. In addition, in contrast to what was demonstrated with IL-10, the levels of TGFβ produced by purified T cell populations from both spleen and PP were elevated to similar or higher levels than those with whole cells, suggesting that T cells and not non-T cells were the source of the TGFβ. This is especially true since DCs are poor producers of TGFβ. Finally, in control studies, of PBS-fed mice treated with anti-IL-12, both spleen and PP cells produced insignificantly higher amounts of TGFβ versus cells from control mice.

Functional Effects of Increased TGF≧Production by Cells From Fed Plus Anti-IL-12 Treated Mice

To determine whether increased TGFβ production by cells from fed plus anti-IL-12 treated mice actively suppresses normal T cell responses, in vitro cell mixing experiments were performed. Since TGFβ is produced relatively late (peak levels after 72-120 hours) in culture (22), proliferation levels were measured after 116 hours in culture, a point when PP responses were suppressed by anti-IL-12 treatment. Cells from both the spleen and the PP of the fed plus anti-IL-12 treated mice were found to induce a marked reduction in the proliferative responses to OVA when mixed in a 1:1 ratio with cells of control mice. Such suppression, however, was not observed when cells of the fed mice were mixed with cells of the control mice. To determine if this suppressor effect was related to TGFβ secretion, in separate studies, similar cell mixing experiments were performed in the presence and absence of anti-TGFβ antibodies and revealed that anti-TGFβ led to an almost complete reversal of suppression. Thus, splenocytes from the control (unfed) mice (baseline proliferation by [³H]-thymidine incorporation, 109,301+5,809 counts per minute, cpm) were mixed at a 1:1 ratio with splenocytes from the fed plus anti-IL-12 treated group (baseline proliferation 4,140+259 cpm, p<0.01 compared to baseline of unfed mice). The suppressed proliferation in the mixed cell cultures (19,940+1299 cpm) was reversed to the values expected (74,249+8107 cpm) if no suppression was occurring, i.e. values similar to the mean proliferation of the two cell populations alone.

In further studies of the possible suppressive effect of TGFβ secretion, the capacity of spleen and PP cells to proliferate in the presence of anti-TGFβ was ascertained. As shown in Table 1, the addition of anti-TGFβ mAb to cultures of cells from the fed plus anti-IL-12 treated mice resulted in a three-fold increase of proliferation, whereas such addition to cultures of cells from the fed-only group did not significantly increase proliferation. Addition of normal chicken Ig (as a control for the anti-TGFβ mAbs) had no effect on proliferative responses. Thus, anti-TGFβ partially reversed the suppressor effect of cells from anti-IL-12 treated mice.

Administration of Anti-IL-12 Induces Increased Apoptosis of Antigen-Specific T Cells in Fed Mice

The fact that proliferative responses in PP cells of fed plus anti-IL-12 treated mice were only partially reversed by anti-TGFβ mAb suggested that mechanisms other than enhanced active suppression were accounting for the effects of anti-IL-12 treatment. One possibility, suggested by the fact that proliferative responses of PP T cells rapidly fall off with time in culture, is that anti-IL-12 treatment leads to increased cell death. Thus, the role of clonal deletion in mice given oral antigen in the presence or absence of anti-IL-12 was investigated.

Using the in situ detection of apoptotic cells by TUNEL technique, the number of apoptotic PP cells after OVA feeding was observed to be increased when compared to PBS-fed mice. An increased number of apoptotic cells after feeding and simultaneous administration of anti-IL-12 was also shown. Finally, the overall number of apoptotic cells in the spleen was demonstrated to be lower than in the PP and no discernable difference could be seen between the treatment groups.

To demonstrate that the apoptotic cells seen with the in situ staining were T cells carrying the OVA-specific TCR, dual color flow cytometry was performed, using the TUNEL technique and the clonotypic antibody KJ1-26. As shown in Table II, the percentage of freshly isolated KJ1-26⁺ apoptotic cells in the PP was greatly increased in fed plus anti-IL-12 treated mice (29.6%) when compared to fed-only (16.4%) and control (7.5%) mice. On the other hand, the rate of apoptosis in the B cell population was similar in these groups. In the spleen, the degree of apoptosis was lower than that in the PP; nevertheless, the spleen contained a similar increase in KJ1-26⁺ apoptotic cells in the OVA-fed plus anti-IL-12 treated group, as compared with the fed-only group. To determine whether the hypoproliferative responses of fed and fed plus anti-IL-12 treated mice were, in fact, due to apoptosis after restimulation in vitro, the TUNEL technique was also performed on cultured cells, demonstrating that the percentage of apoptotic PP and spleen cells was indeed negatively correlated with the proliferative responses (Table II). Thus, since the highest percentage of apoptotic cells over time was found in the fed plus anti-IL-12 treated group, the neutralization of IL-12 appeared to have primed cells for activation-induced apoptosis (i.e., after restimulation in vitro).

Administration of oral antigen and antibodies to IL-12 to a human subject having an autoimmune disease, allergic disease, GvH disease or transplantation rejection. To enhance oral tolerance in a human subject, or to treat or prevent an autoimmune disease, allergic disease, GvH disease or transplantation rejection in a human subject, 0.01-100 mg/kg of antigen can be administered orally to the subject and 1-100 mg/kg of antibodies to IL-12 can be administered parenterally one time each week over a two year period or until clinical parameters, i.e, signs, symptoms and objective laboratory tests with which clinicians in this field will be familiar, indicate prolonged remission, stabilization or improvement. For oral administration of antibodies to IL-12, 500 to 1000 mg of antibodies to IL-12 can be administered in a single dose each day for up to two years or until various clinical parameters known to field clinicians in this field, i.e., signs, symptoms and objective laboratory tests, indicate prolonged remission, stabilization or improvement.

Production of humanized mouse antibodies to IL-12. Rodent monoclonal or polyclonal antibodies can be modified according to the protocols set forth in Junghans et al. (50), Brown et al. (51) and Kettleborough et al. (52). Specifically, rodent antibodies can be modified for human administration by constructing, through recombinant DNA protocols known to one of skill in the art, a chimeric rodent-human antibody composed of rodent variable regions and human heavy and light chain constant regions. Another approach to humanizing rodent antibodies is to graft rodent complementarity-determining regions (CDRs) from the rodent variable regions into human variable regions. By using either of these approaches, rodent antibodies can be humanized for administration into human subjects.

Throughout this application, various publications are referenced The disclosures of these publications in their entireties are hereby incorporated by reference into this application in order to more fully describe the state of the art to which this invention pertains.

Although the present invention has been described with reference to specific details of certain embodiments thereof, it is not intended that such details should be regarded as limitations upon the scope of the invention except as and to the extent that they are included in the accompanying claims.

References

1. Mowat, A. Mcl. 1994. Oral tolerance and regulation of immunity to dietary antigens. In Handbook of Mucosal Immunology. P. L. Ogra, J. R. McGhee, J. Mestecky, W. Strober, and J. Bienenstock, editors. Academic Press, San Diego, Calif. pp. 185-201.

2. Weiner, H. L., A. Friedman, A. Miller, S. J. Khoury, A. Al-Sabbagh, L. Santos, M. Sayegh, R. B. Nussenblatt, D. E. Trentham, and D. A. Hafler. 1994. Oral tolerance: Immunologic mechanisms and treatment of animal and human organ-specific autoimmune diseases by oral administration of autoantigens. Annu. Rev. Immunol. 12:809-837.

3. Nagler-Anderson, C., L. A. Bober, M. E. Robinson, G. W. Siskind, and G. J. Thorbecke. 1986. Suppression of type II collagen-induced arthritis by intragastric administration of soluble type II antigen. Proc. Natl. Acad. Sci. USA 83:7443-7446.

4. Whitacre, C. C., I. E. Gienapp, C. G. Orosz, and D. M Bitar. 1991. Oral tolerance in experimental autoimmune encephalitis. III. Evidence for clonal anergy. J. Immunol. 147:2155-2163.

5. Higgins, P. J., and H. L. Weiner. 1988. Suppression of experimental autoimmune encephalomyelitis by oral administration of myelin basic protein and its fragments. J. Immunol. 140:440-445.

6. Nussenblatt, R. B., R. R. Caspi, R. Mahdi, C. -C. Chan, F. Roberge, O. Lider, and H. L. Weiner. 1990. Inhibition of S-antigen induced experimental autoimmune uveoretinitis by oral induction of tolerance with S-antigen. J. Immunol. 144:1689-1696.

7. Weiner, H. L., G. A. Mackin, M. Matsui, E. J. Orav, S. J. Khoury, D. M. Dawson, and D. A. Hafler. 1993. Double-blind pilot trial of oral tolerization with myelin antigens in multiple sclerosis. Science 259:1321-1324.

8. Trentham, D. E., R. A. Dynesius-Trentham, E. J. Orav, D. Combitchi, C. Lorenzo, K. L. Sewell, D. A. Hafler, and H. L. Weiner. 1993. Effects of oral administration of collagen on rheumatoid arthritis. Science 261:1727-1730.

9. Friedman, A., and H. L. Weiner. 1994. Induction of anergy or active suppression following oral tolerance is determined by antigen dosage. Proc. Natl. Acad. Sci. USA 91:6688-6692.

10. Chen, Y., J. -I. Inobe, R. Marks, P. Gonella, V. K. Kuchroo, and H. L. Weiner. 1995. Peripheral deletion of antigen-reactive T cells in oral tolerance. Nature 376:177-180.

11. Khoury, S. J., W. W. Hancock, and H. L. Weiner. 1992. Oral tolerance to myelin basic protein and natural recovery from experimental autoimmune encephalomyelitis are associated with downregulation of inflammatory cytokines and differential upregulation of transforming growth factor β, interleukin 4, and prostaglandin E expression in the brain. J. Exp. Med. 176:1355-1364.

12. Chen, Y., J. -I. Inobe, and H. L. Weiner. 1995. Induction of oral tolerance to myelin basic protein in CD8-depleted mice: Both CD4+ and CD8+ cells mediate active suppression. J. Immunol. 155: 910-916.

13. Miller, A., O. Lider, A. B. Roberts, M. B. Sporn, and H. L. Weiner. 1992. Suppressor T cells generated by oral tolerization to myelin basic protein suppress both in vitro and in vivo immune responses by the release of transforming growth factor β after antigen-specific triggering. Proc. Natl. Acad. Sci. USA. 89:421-425.

14. Chen, Y., V. K. Kuchroo, J. -I. Inobe, D. A. Hafler, and H. L. Weiner. 1994. Regulatory T cell clones induced by oral tolerance: Suppression of autoimmune encephalitis. Science 265:1237-1240.

15. Miller, A., O. Lider, and H. L. Weiner. 1991. Antigen-driven bystander suppression after oral administration of antigen. J. Exp. Med. 174:791-798.

16. Santos, L. M. B., A. Al-Sabbagh, A. Londono, and H. L. Weiner. 1994. Oral tolerance to myelin basic. protein induces regulatory TGF-β secreting cells in Peyer's patches of SJL mice. Cell. Immunol. 157:439-447.

17. Murphy, K. M., A. B. Heimberger, and D. Y. Loh. 1990. Induction by antigen of intrathymic apoptosis of CD4⁺CD8⁺TCR^{lo} thymocytes in vivo. Science 250:1720-1724.

18. Crowley, M. K., M. Inaba, M. Wittmer-Pack, and R. M. Steinman. 1989. The surface of mouse dendritic cells: FACS analysis of dendritic cells from different tissues including thymus. Cell. Immunol. 118:108-125.

19. Flaumenhaft, R., S. Kojima, M. Abe, and D. B. Rifkin. 1993. Activation of latent transforming growth factor β. Adv. Pharmacol. 24.51-76.

20. Haskins, K., R. Kubo, J. White, M. Pigeon, J. Kappler, and P. Marrack. 1983. The major histocompatibility complex-restricted antigen receptor on T cells. I. Isolation with a monoclonal antibody. J. Exp. Med. 157:1149-1169.

21. Trinchieri, G. 1995. Interleukin-12: a proinflammatory cytokine with immunoregulatory functions that bridge innate resistance and antigen-specific adaptive immunity. Annu. Rev. Immunol. 13:251-276.

22. Kehrl, J. H., L. M. Wakefield, A. B. Roberts, S. Jakolew, M. Alvarez-Mon, R. Derynck, M. B. Sporn, and A. S. Fauci. 1986. Production of transforming growth factor β by human T lymphocytes and its potential role in the regulation of T cell growth. J. Exp Med. 163:1037-1050.

23. Kelsall, B. L., Ehrhardt, R. O, and W. Strober. 1994. Induction of a primary interferon-γ response by Peyer's patch dendritic cells is mediated by IL-12 and inhibited by IL-4. FASEB J 8.A207 (Abstr.).

24. Taguchi, T., J. R. McGhee, R. L. Coffman, K. W. Beagley, J. H. Eldridge, K. Takatsu, and H. Kiyono. 1990. Analysis of Th1 and Th2 cells in murine gut-associated tissues. Frequencies of CD4+ and CD8+ T cells that secrete IFN-γ and IL-5. J. Immunol. 145:68-75.

25. Xu-Amano, J., H. Kiyono, R. J. Jackson, H. F. Staats, K. Fujihashi, P. D. Burrows, C. O. Elson, S. Pillai, and J. R. McGhee. 1993. Helper T cell subsets for immunoglobulin A responses: oral immunization with tetanus toxoid and cholera toxin as adjuvant selectively induces Th2 cells in mucosa associated tissues. J. Exp. Med. 178:1309-1320.

26. Wilson, D. A., M. Bailey, N. A. Williams, and C. R. Stokes. 1991. The in vitro production of cytokines by mucosal lymphocytes immunized by oral administration of keyhole limpet hemocyanin using cholera toxin as an adjuvant. Eur. J. Immunol. 21:2333-2339.

27. Vajdy, M., and N. Lycke. 1993. Stimulation of antigen-specific T- and B-cell memory in local as well as systemic lymphoid tissues following oral immunization with cholera toxin adjuvant. Immunology 80:197-203.

28. Kearney, E. R., K. A. Pape, D. Y. Loh, and M. K. Jenkins. 1994. Visualization of peptide-specific T cell immunity and peripheral tolerance induction in vivo. Immunity 1: 327-339.

29. Kelsall, B. L. and W. Strober. 1995. Distinct populations of dendritic cells are present in the subepithelial dome and T cell regions of the murine Peyer's patch. J. Exp. Med. 183:237-247.

30. Philips-Quagliata, J. M., and M. E. Lamm. 1994. Lymphocyte homing to mucosal effector sites. In. Handbook of Mucosal Immunology. P. L. Ogra, J. R. McGhee, J. Mestecky, W. Strober, and J. Bienenstock, editors. Academic Press, San Diego, Calif. pp. 225-239.

31. Rocha, B., A. Grandien, and A. A. Freitas. 1995. Anergy and exhaustion are independent mechanisms of peripheral T cell tolerance. J. Exp. Med. 181.993-1000.

32. Schmitt, E., P. Hoeln, C. Huels, S. Goedert, N. Palm, E. Ride, and T. Germann. 1994. T helper type 1 development of naive CD4⁺ T cells requires the coordinate action of interleukin-12 and interferon-γ and is inhibited by transforming growth factor-β. Eur. J. Immunol. 24:793-798.

33. Barral-Netto, M., A. Barral, C. E. Brownell, Y. A. Skeiky, L. R. Ellingsworth, D. R. Twardzik, and S. G. Reed. 1992. Transforming growth factor-β in leishmanial infection: a parasite escape mechanism. Science 257: 545-548.

34. Clerici, M., A. Sarin, R. L. Coffman, T. A. Wynn, S. P. Blatt, C. W. Hendrix, S. F. Wolf, G. M. Shearer, and P. A. Henkart. 1994. Type 1/type 2 cytokine modulation of T-cell programmed cell death as a model for human immunodeficiency virus pathogenesis. Proc. Natl. Acad. Sci. USA 91: 11811-11815.

35. Kobayashi, M., L. Fitz, M. Ryan, R. M. Hewick, S. C. Clark, S. Chan, R. Loudon, F. Sherman, B. Perussia, and G. Trinchieri. 1989. Identification and purification of Natural Killer Cell stimulatory factor (NKSF), a cytokine with multiple biologic effects on human lymphocytes. J Exp Med 170: 827-845.

36. Weller, M., D. B. Constam, U. Malipiero, and A. Fontana. 1994. Transforming growth factor-β2 induces apoptosis in murine T cell clones without down-regulating bcl-2 mRNA expression. Eur. J. Immunol. 24:1293-1300.

37. Ramsdell, F., M. S. Seaman, R. E. Miller, K. S. Picha, M. K. Kennedy, and D. H. Lynch. 1994. Differential ability of Th1 and Th2 T cells to express Fas ligand and to undergo activation-induced cell death. Int. Immunol. 6:1545-1553.

38. Neurath, M. F., I. Fuss, B. L. Kelsall, E. Stüber, and W. Strober. 1995. Antibodies to IL-12 abrogate established experimental colitis in mice. J. Exp. Med. 182: 1281-1290.

39. Leonard, J. P., Waldburger, K. E., and S. J. Goldman. 1995. Prevention of experimental autoimmune encephalomyelitis by antibodies against interleukin 12. J. Exp. Med. 181:381-386.

40. Kobayashi, M., L. Fitz, M. Ryan, R. M. Hewick, S. C. Clark, S. Chan, R. Loudon, F. Sherman, B. Perussia, and G. Trinchieri. 1989. Identification and purification of natural killer cell stimulatory factor (NKSF), a cytokine with multiple biological effects on human lymphocytes. J. Exp. Med. 170:827-845.

41. Seder, R. A., R. Gazzinelli, A. Sher, and W. E. Paul. 1993. IL-12 acts directly on CD4+T cells to enhance priming for IFN-γ production and diminishes IL-4 inhibition of such priming. Proc. Natl. Acad. Sci. USA 90:10188-10192.

42. Ling, P., M. K. Gately, U. Gubler, A. S. Stern, P. Lin, K. Hollfelder, C. Su, Y. -C. E. Pan, and J. Hakimi. 1995. Human IL-12 p40 homodimer binds to the IL-12 receptor but does not mediate biologic activity. J. Immunol. 154:116-127.

43. Podlaski, F. J., V. B. Nanduri, J. D. Hulmes, Y. -C. E. Pan, W. Levin, W. Danho, R. Chizzonite, M. K. Gately, and A. S. Stem. 1992. Molecular characterization of interleukin 12. Arch. Biochem. Biophys. 294:230-237.

44. Kubin, M., M. Kamoun, and G. Trinchieri. 1994. Interleukin 12 synergizes with B7/CD28 interaction in inducing efficient proliferation and cytokine production of human T cells. J. Exp. Med. 180:211-222.

45. Wynn, T. A., I. Eltoum, I. P. Oswald, A. W. Cheever, and A. Sher. 1994. Endogenous interleukin 12 (IL-12) regulates granuloma formation induced by eggs of Schistosoma mansoni and exogenous IL-12 both inhibits and prophylactically immunizes against egg pathology. J. Exp. Med. 179:1551-1561.

46. Murray, H. W., and J. Hariprashad. 1995. Interleukin 12 is effective treatment for an established systemic intracellular infection: experimental visceral leishmaniasis. J. Exp. Med. 181:387-391.

47. Eldridge et al. 1989. Cur. Topics in Microbiol. and Immunol.; 146:59-65.

48. Harlow and Lane, Antibodies; A Laboratory Manual, Cold Spring Harbor Laboratory, Cold Spring Harbor, N.Y., (1988).

49. Martin, E. W. Remington's Pharmaceutical Sciences, Martin, latest edition, Mack Publishing Co., Easton, Pa.

50. Junghans et al. 1990. Cancer Research 50:1495-1502.

51. Brown et al. 1991. Proc. Natl. Acad. Sci. USA 88:2663-2667.

52. Kettleborough et al. 1991. Protein Engineering 4:773-783.

53. Oka et al. 1990. Vaccine, 8:573-576.

54. Aramaki et al., 1994. Induction of oral tolerance after feeding of ragweed pollen extract in mice. Immunol. Lett. 40:21-5.

55. Bjorksten et al., 1986. Clinical and immunological effects of oral immunotherapy with a standardized birch pollen extract. Allergy 41:290-295.

56. Enomoto et al., 1993. Milk whey protein fed as a constituent of the diet induced both oral tolerance and a systemic humoral response, while head-denatured whey protein induced only oral tolerance. Clin. Immunol. Immunopathol. 66:136-42.

57. Holt et al., 1987. Suppression of IgE responses following inhalation of antigen. A natural homeostatic mechanism which limits sensitization to aeroallergens. Immunol. Today 8:14-15.

58. Sosroseno, W., 1995. A review of the mechanisms of oral tolerance and immunotherapy. J. Royal Soc. Med.-88-14-17.

59. Sosroseno, W, 1995. The immunology of nickel-induced allergic contact dermatilis. Asian Pac. J. Allergy Immunol. 13:173-81.

60. Van Hoogstraten et al., 1992. Persistent immune tolerance to nickel and chromium by oral administration prior to cutaneous sensitization. J. Invest Derm. 99:608-616.

61. Hancock et al., 1992. Oral but not intravenous alloantigen prevents accelerated allograft rejection by selective intracraft Th2 cell activation. Transplantation 55:1 112-1118.

62. Hrstka et al., 1992. Prolongation of islet allograft survival following immunologic conditioning by antigen in the pig. Transplant Proc. 24:663-664.

63. Sayegh et al., 1992. Down-regulation of immune response to histocompatibility antigens and prevention of sensitization by skin allografts by orally administered alloantigen. Transplantation 53:163-166.

64. Sayegh et al., 1992. Induction of immunity and oral tolerance with polymorphic class II major histocompatibility complex allopeptides in the rat. Proc. Natl. Acad. Sci. USA 89:7762-7766.

65. Liote et al., 1996. Inhibition and prevention of monosodium urate monohydrate crystal-induced acute inflammation in vivo by transforming growth factor beta 1. Arthritis Rheum. 39:1192-8.

69. Sosroseno, W., 1995. A review of the mechanisms of oral tolerance and immunotherapy. J. R. Soc. Med. 88:14-7.

70. Friedman et al., 1994. Oral tolerance: a biologically relevant pathway to generate peripheral tolerance against external and self antigens. Chem. Immunol. 58:259-90.

71. Mestecky et al., 1996. Induction of tolerance in humans: effectiveness of oral and nasal immunization routes. Ann N. Y. Acad. Sci. 778:194-201.

72. Husby et al., 1994. Oral tolerance in humans. T cell but not B cell tolerance after antigen feeding. J. Immunol. 152:4663-70.

73. Hohol et al., 1996. Three-year open protocol continuation study of oral tolerization with myelin antigens in multiple sclerosis and design of a phase III pivotal trial. Ann. N. Y. Acad. Sci. 778:243-50.

74. Weiner et al., 1993. Double-blind pilot trial of oral tolerization with myelin antigens in multiple sclerosis. Science 259:1321-4.

75. Trentham et al., 1993. Effects of oral administration of type II collagen on rheumatoid arthritis. Science 261:1727-30.

76. Van Oosten et al., 1995. Multiple sclerosis therapy. A practical guide. Drugs 49:200-12.

77. Rolak, L. A., 1996. The diagnosis of multiple sclerosis. Neurol. Clin. 14:27-43.

78. Kardon, E. M., Acute asthma. Emerg. Med. Clin. North Am. 14:93-114.

79. Martin, R. J., 1994. Management of patients with chronic asthma: controversies and future directions. Ann. Allergy 72:390-2.

80. Powell, C. V., 1993. Management of acute asthma in childhood. Br. J. Hosp. Med. 50:272-5.

81. Lynch et al., 1996. Lung transplantation in chronic airflow limitation. Med. Clin. North Am. 80:657-70.

82. Corris, P. A., 1995. Post heart/lung transplantation management. J. R. Soc. Med. 25:37-40.

83. Engeler, C. E., 1995. Heart-lung and lung transplantation.” Radiol. Clin. North Am. 33:559-80.

84. Davis et al., 1995. Pulmonary transplantation Ann. Surg. 221:14-28.

85. Kremer, J. M., 1995. The changing face of therapy for rheumatoid arthritis.”Rheum. Dis. Clin. North Am. 21:845-52.

86. Dawes et al., 1995. Treatment of early rheumatoid arthritis: a review of current and future concepts and therapy. Clin. Exp. Rheumatol. 13:381-94.

87. Porter et al., 1993. Medical management of rheumatoid arthritis. B. M. J. 307:425-8.

88. Jaffe, I. A., 1992. New approaches to the management of rheumatoid arthritis. J. Rheumatol. 36:2-8.

89. Wilke et al., 1991. Therapy for rheumatoid arthritis: combinations of disease-modifying drugs and new paradigms of treatment. Semin. Arthritis Rheum. 21:21-34

TABLE I
Addition of anti-TGFβ in vitro partially reverses
suppression of proliferative responses

| [3H] Thymidine | PBS fed | | OVA fed + |
|---|---|---|---|
| incorporation (cpm) | control | OVA fed | anti-IL-12 |
| Peyer's patches | | | |
| Baseline (116 hours) | 31571 ± 2809 | 8752 ± 1287** | 3503 ± 943*** |
| + anti-TGFβ | 22712 ± 1519 | 7691 ± 432** | 10398 ± 2101* |
| Spleen | | | |
| Baseline (116 hours) | 32084 ± 2318 | 11271 ± 1523* | 7757 ± 465*** |
| + anti-TGFβ | 26217 ± 975 | 12416 ± 886* | 25647 ± 2546 |

Proliferative response of cells from the PP and the spleen from PBS fed control mice, from OVA fed mice (3×250 mg OVA), and from OVA fed plus anti-IL-12 treated mice were determined in triplicates after stimulation with OVA (1 mg/ml) in vitro at 116 hours of culture (=baseline) and these responses were compared with cultures in which anti-TGFβ (10 μg/ml) or, as control, normal chicken Ig (10 μg/ml) had also been added at the initiation of the culture. Proliferative responses with or without addition of chicken Ig were similar. Data represent the mean of triplicates (±SD). One of two experiments performed is shown.

* indicates p<0.05 vs. control, ** p<0.01, and *** p<0.001 vs. control

TABLE II
Quantitation of apoptotic cells from the
PP and the spleen by TUNEL technique

| | | | Fed + |
|---|---|---|---|
| % Apoptotic cells for | Control | Fed | anti-IL-12 |
| Peyer's patches | | | |
| Freshly isolated cells | | | |
| KJ1-26 + cells | 7.45 | 16.37 | 29.61 |
| B220⁺ cells | 6.00 | 11.79 | 10.04 |
| All cells | 12.49 | 23.47 | 34.32 |
| Cultured cells (all cells) | | | |
| 92 hours of culture | 14.8 | 34.8 | 70.9 |
| 116 hours | 24.4 | 71.7 | 74.6 |
| 164 hours | 33.4 | 42.3 | 69.7 |
| Spleen | | | |
| Freshly isolated cells | | | |
| KJ1-26 + cells | 1.72 | 2.98 | 6.02 |
| B220⁺ cells | 2.13 | 4.56 | 3.99 |
| All cells | 3.48 | 5.92 | 8.80 |
| Cultured cells (all cells) | | | |
| 92 hours of culture | 11.9 | 21.2 | 30.7 |
| 116 hours | 7.6 | 11.3 | 16.7 |
| 164 hours | 10.8 | 15.8 | 18.3 |

Cells from the PP and the spleen were prepared as described from control mice, mice fed OVA (3×250 mg), and mice fed plus treated with anti-IL-12. Dual color flow cytometry using clonotypic mAb KJ1-26 or mAb to B cells (B220) followed by fluorescent anti-digoxigenin antibody (which detects degraded DNA typical for apoptotic cells), or the latter antibody alone (“all cells”) was performed on freshly isolated or cultured cells (stimulated with 1 mg/ml OVA in vitro). Data represent the mean of duplicates of percentage apoptotic cells.

What is claimed is:

1. A method for enhancing oral tolerance to an antigen associated with an autoimmune disease in a subject having the autoimmune disease comprising orally administering to the subject an antigen associated with the autoimmune disease and administering an inhibitor of interleukin-12 in amounts sufficient to enhance oral tolerance.

2. The method of claim 1, wherein the inhibitor of interleukin-12 is an antibody specifically reactive with interleukin-12.

3. The method of claim 2, wherein the antibody is monoclonal.

4. The method of claim 1, wherein the subject is a human.

5. The method of claim 1, wherein the autoimmune disease is multiple sclerosis.

6. The method of claim 1, wherein the autoimmune disease is Crohn's disease.

7. The method of claim 1, wherein the autoimmune disease is rheumatoid arthritis.

8. The method of claim 1, wherein the autoimmune disease is psoriasis.

9. The method of claim 1, wherein the autoimmune disease is diabetes mellitus.

10. The method of claim 1, wherein the autoimmune disease is from the group consisting of ulcerative colitis, pernicious anemia, autoimmune gastritis, Bechet's disease, idiopathic thrombocytopenic purpura, Wegener's granulomatosis, autoimmune polyendocrinopathies, Still's disease, Lambert-Eaton myasthenia syndrome, myasthenia gravis, Goodposture's syndrome, autoimmune orchitis, autoimmune uveitis, systemic lupus erythematosus, Sjogren's syndrome and ankylosing spondylitis.

11. A method for treating or preventing an autoimmune disease in a subject comprising orally administering to the subject an antigen associated with the autoimmune disease and administering an inhibitor of interleukin-12 in amounts sufficient to treat or prevent the autoimmune disease, thereby treating or preventing the autoimmune disease.

12. The method of claim 11, wherein the inhibitor of interleukin-12 is an antibody specifically reactive with interleukin-12.

13. The method of claim 12, wherein the antibody is monoclonal.

14. The method of claim 11, wherein the subject is a human.

15. The method of claim 11, wherein the autoimmune disease is multiple sclerosis.

16. The method of claim 11, wherein the autoimmune disease is Crohn's disease.

17. The method of claim 11, wherein the autoimmune disease is rheumatoid arthritis.

18. The method of claim 11, wherein the autoimmune disease is psoriasis.

19. The method of claim 11, wherein the autoimmune disease is diabetes mellitus.

20. The method of claim 11, wherein the autoimmune disease is from the group consisting of ulcerative colitis, pernicious anemia, autoimmune gastritis, Bechet's disease, idiopathic thrombocytopenic purpura, Wegener's granulomatosis, autoimmune thyroiditis, autoimmune oophoritis, bullous pemphigoid, pemphigus, polyendocrinopathies, Still's disease, Lambert-Eaton myasthenia syndrome, myasthenia gravis, Goodposture's syndrome, autoimmune orchitis, autoimmune uveitis, systemic lupus erythematosus, Sjogren's syndrome and ankylosing spondylitis.
